(19) United States
(12) Patent Application Publication
(10) Pub. No.: US 2022/0370530 A1
(43) Pub. Date: Nov. 24, 2022

(54) PROCESS AND APPARATUS FOR MULTI-PHASE EXTRACTION OF ACTIVE SUBSTANCES FROM BIOMASS

(71) Applicant: LEHUA GROUP USA, INC., Bellevue, WA (US)

(72) Inventor: Randy REED, King City, CA (US)

(21) Appl. No.: 17/771,380

(22) PCT Filed: Oct. 23, 2020
(86) PCT No.: PCT/US2020/057214

§ 371 Date: Apr. 22, 2022

Related U.S. Application Data
(60) Provisional application No. 62/925,137, filed on Oct. 23, 2019.

(51) Int. Cl. A61K 36/185; B01D 11/02; A23L 33/105

(52) U.S. Cl. CPC A61K 36/185; B01D 11/0203; B01D 11/0288; A23L 33/105; B01D 11/0284; A23V 2002/00; A61K 2236/17; A61K 2236/37; A61K 2236/39

(57) ABSTRACT

A method for performing a multi-phase extraction of plant biomass to obtain active substances is provided. The method uses a novel freeze-dry extraction technique to obtain a volatile terpene fraction from frozen plant material. In particular, the method disclosed provides terpene extracts with compositions that strongly resemble that of the parent plant material.

### BACKGROUND

### Technical Field

This disclosure relates to methods, processes and apparatus for isolating fractions of natural products from plant materials.

### Description of the Related Art

Whole plant-like extracts that maintain the balance of natural products as they existed in the raw material are highly desired by consumers of products derived from such extracts. This is particularly evident for products produced from plants of the genera Cannabis and Humulus (marijuana/hemp and hop, respectively). The interest in more native plant-like extracts is driven by a couple factors. First, consumers prefer the natural flavor and aroma profiles which mimic the sensory experience from consuming the whole plant. Secondly, there is a significant drive to try and maintain the entirety of the natural product profiles because the therapeutic benefits derived from the biologically active compounds could be enhanced or cooperatively modulated by combining the biologically active components (e.g., cannabinoids) of the native plants; a phenomenon known in the Cannabis industry as the entourage effect. Non-cannabinoids such as terpenoids and flavonoids may also take part in the entourage effect.

Conventional methods for extracting natural products from plant biomass show significant limitations in their ability to maintain native natural product profiles, particularly for volatile or unstable components such as terpenoids, as these compounds can be easily lost due to the extraction conditions (e.g. through evaporation) or they chemically change (e.g., degradation) to different species.

For instance, there are numerous ways to commercially extract terpenes from biomass but they all cause changes to the native natural product profiles, particularly for the more reactive and volatile terpene components, which are susceptible to loss due to chemical conversion upon heating and/or exposure to oxygen or other reactive chemical species.

Accordingly, there remains a need for processes whereby extracts from plants maintain their native natural product profiles, such that the products derived from those products display the aroma, flavor, and medicinal qualities resembling those of the native plant material.

### BRIEF SUMMARY

Various embodiments of the present disclosure provide processes for fractionally obtaining extracts of naturally-occurring chemical substances from plant materials. Advantageously, the processes disclosed herein address the need in the art for preserving the natural profile of the chemical substance as they exist in the native plant from which the extracts are obtained.

One embodiment provides a process comprising: providing plant biomass; and lyophilizing the plant biomass to provide desiccated plant biomass and a condensate including water and one or more volatile substances having a boiling point of no more than 250° C. at atmospheric pressure.

In preferred embodiments, the plant biomass is frozen, or even more preferably, fresh-frozen. In a more specific embodiment, the fresh-frozen plant biomass is obtained by flash freezing fresh plant biomass, such as the whole flowers of cannabis, hemp or hop.

In other embodiments, the fresh-frozen plant biomass are provided in the form of fine particles prior to lyophilizing. The fine particles can be obtained by milling, grinding or otherwise mechanically agitated at below freezing temperature, e.g., 0° C. or lower, −10° C. or lower, −20° C. or lower, or −30° C. or lower.

Although fresh-frozen plant biomass is preferred, the process disclosed herein is also applicable to fresh plant biomass or cured plant biomass (e.g, fresh plant biomass that has been dried in ambient temperature for an extended period of time).

In another specific embodiment, lyophilizing the plant biomass (e.g., fresh-frozen plant biomass) comprises running one or more cycles of sublimation and freezing, each cycle including: sublimating solid water in the fresh-frozen plant biomass under vacuum and heating to a temperature of no more than 65° C. to provide sublimated plant biomass; and freezing the sublimated plant biomass, wherein the cycle is repeated till the sublimated plant biomass has a moisture level (i.e., water content) of no more than 2% w/w, thereby providing the desiccated plant biomass.

In particular, the condensate includes non-polar terpenes, and an aqueous mixture having polar terpenes, plant saccharides, esters, phenols or combination thereof. The aqueous mixture may be subsequently separated from the non-polar terpenes.

In other embodiments, the process further comprising contacting the desiccated plant biomass with an extracting medium (e.g., supercritical or subcritical CO₂) under conditions sufficient to sequentially extract a first fraction of one or more terpenes; a second fraction of neutral cannabinoids (e.g., THC, CBD or a combination thereof); and a third faction of acidic cannabinoids (THCA, CBDA or a combination thereof).

In alternative embodiments, the process further comprising contacting the desiccated plant biomass with an extracting medium such as ethanol under conditions sufficient to extract neutral and/or acidic cannabinoids from the desiccated plant biomass.

### BRIEF DESCRIPTION OF THE SEVERAL VIEWS OF THE DRAWINGS

Exemplary embodiments are illustrated in referenced figures. It is intended that the embodiments and figures disclosed herein are to be considered illustrative rather than restrictive.

FIG. 1 depicts a flow diagram of a general process according to an embodiment of the present disclosure

FIG. 2 depicts a flow diagram of a process for extracting volatile components according to an embodiment of the present disclosure.

FIG. 3 depicts a flow diagram of a process for fractional extraction using supercritical or subcritical CO₂ according to an embodiment of the present disclosure.

FIG. 4 depicts the terpene finger print (chemovar) in a bar-chart representation of the gas chromatography mass spectrometry (GC/MS) analysis of the extract of the present invention; said extract derived from the Tesla Tower Cannabis cultivar (top frame). For comparison, the bottom frame depicts the chemovar as determined from the native plant material.

FIG. 5 depicts the terpene finger print (chemovar) in a bar-chart representation of the gas chromatography mass spectrometry (GC/MS) analysis of terpene extracts from the Tesla Tower Cannabis cultivar (top frame). For comparison, the bottom frame depicts the chemovar as determined from the native plant material.

FIGS. 6 and 7 depict a comparison of the early eluting terpene fingerprints (more volatile/lower boiling point) for the present extraction method (FIG. 6) and the hydrocarbon based method (FIG. 7).

### DETAILED DESCRIPTION

The present disclosure is related to processes for extracting from plant biomass an array of chemical substances, including essential oils, bioactive substances and the like. In particular, the combined extracts of the chemical substances resemble their natural profile as they exist in a given plant variety.

Plant varieties, also referred to herein as “cultivars,” are plants that have been produced through cultivation activities and selective breeding practices directed toward desired characteristics. Cultivars, also known as strains, breeds or types, are thus phenotypically different plants and can vary in their appearance, smell, yields, and pharmacological effects. Although the processes disclosed herein are particularly applicable to cultivars of the genera Cannabis (marijuana/hemp) and Humulus (hop), other plant varieties are also contemplated.

The varieties of the chemical substances extracted from a given cultivar are referred to as “chemovar.” Native chemovars are thus the natural compositions or profiles of the chemical varieties as they exist in cultivars. Extracts from cultivars often deviate from the native chemovars due to loss of volatile components, degradation (including oxidation) of reactive substances, incomplete or inadequate extraction. The changes in the extracts, also called “chemovar drift,” thus lower the quality or grade of the extracts. The process disclosed herein addresses the technical problem of “chemovar drift” by preserving the native composition of chemical substances of cultivars, including the most volatile components.

### Cannabis Chemovars

According to the botanical classification of cannabis, there are three main types of cultivars, namely, sativa, indica, hybrids and Ruberallis. Each type has its own numerous and diverse subtypes of various physical characteristics. Although all are used for medicinal or recreational purposes, sativas are generally known for an invigorating and energizing effect such as “head high,” whereas indicas are known for a relaxing full-body effect that can reduce pain and nausea. Hybrids of sativas and indicas have been bred and grown to target specific effects.

Cultivars are, however, inaccurate classifications because they do not correlate to well-defined or reproducible chemical profiles. Rather, the constituent chemical varieties (i.e., chemovars) of a given cannabis are more accurate and reliable indicators of its cultivar effect.

The overall biological effects produced by individual cultivars thus depend on their respective compositions of chemical substances which includes mainlycannabinoids and terpenes, as further described herein.

1. Cannabinoids

Cannabinoids are primarily responsible for the majority of biological effects produced by cannabis. There are more than 100 known cannabinoids, many of which are considered active pharmaceutical ingredients (API). While structurally diverse, they all act on the cannabinoid receptors (e.g., CB1 and CB2 receptors), which are located throughout the body and are involved in a variety of physiological processes including appetite, pain-sensation, mood, and memory.

“Tetrahydrocannabinol,” “THC,” or Δ⁹-THC is the primary psychoactive cannabinoid component of Cannabis. THC acts on CB1 receptors, which are mostly in the brain, and provides the psychoactive “high” experienced by users. THC also provides therapeutic effects that target conditions such as pain, muscle spasticity, glaucoma, insomnia, low appetite, nausea and anxiety.

“THCa” is the non-psychoactive, carboxylic acid form of THC and can be converted to THC by decarboxylation under thermal, light or alkaline conditions. Dab forms of Cannabis extracts can contain high levels of THCa that convert to THC upon vaporizing, thus providing the psychoactive THC to the user.

“Cannabidiol” or “CBD” is a neutral (uncharged), non-psychoactive cannabinoid component of Cannabis. In combination with THC, CBD demonstrates some modulatory properties to reduce adverse THC effects. CBD is also known to possess its own therapeutic properties, notably for the treatment of seizures and most recently autism. CBD can also benefit those experiencing nausea, inflammation and anxiety due to its antidepressant and neuroprotective effects.

“CBDA” is the carboxylic acid form of CBD and can be converted to CDB by decarboxylation under thermal, light or alkaline conditions.

2. Terpenes

Plant terpenes are naturally occurring hydrocarbons of diverse structures. The terpene profiles are produced in intricate proportions based on genomics and environmental conditions. These profiles give each chemovar or chemical “fingerprint” its unique aroma and flavor attributes. In addition, some terpenes are believed to modulate the pharmacological effects of cannabinoids.

Terpenes are biosynthetically constructed by isoprene units. Unless otherwise specified, the term “terpenes” encompasses terpene derivatives or “terpenoids,” which are hydrocarbon terpenes with oxygen-containing functional groups. Plant terpenes may be classified by the number of isoprene units such as hemiterpenes (a single isoprene unit, often functionalized), monoterpene (two isoprene units), sesquiterpenes (three isoprene units), diterpenes (four isoprene units), and so on. Common cannabis terpenes include, for example, bisabolol, caryophyllene, eucalyptol, linalool, myrcene, ocimene, pinene, limonene, humulene, and terpinolene, etc.

Terpenes are often formulated with cannabinoids extracts because they bring flavors as well as act as a diluent to the more viscous cannabinoids such as THC or CBD oils (e.g., for vaping pens). In addition, terpenes modulate the effects of cannabinoids and contribute to the more efficacious “entourage” effect.

Conventionally, in an effort to mimic the native chemovar profiles, formulators take a native Cannabis GC-MS chromatogram and construct a terpenoid profile with terpenoids from non-Cannabis botanicals. For instance, limonene is produced in lemons, alpha pinene is produced in conifers, linalool is produced in lavender, and beta-caryophyllene is produced in black pepper. Many of the aforementioned terpenes are readily available from commercial sources; however, some terpenoids produced by Cannabis are not readily available to non-native terpene formulators. This not only limits the formulator but also limits the capabilities to reference a standard in analytical instrumentation and thus putting the formulator at an even greater molecular disadvantage. There are over 500 terpenoids in cannabis. Thus, manmade Cannabis mimicking profiles are not nearly as efficacious and the sensory experience inferior to cannabis-derived terpenes (CDT).

CDTs are thus desirable extracts because they preserve the natural terpene profile of a given cultivar. However, extraction of CDTs is challenging because they are present in a small amount of the plant total mass (less than 5%) and can often be lost during extraction due to their volatility and reactivity.

### Fractional Extraction

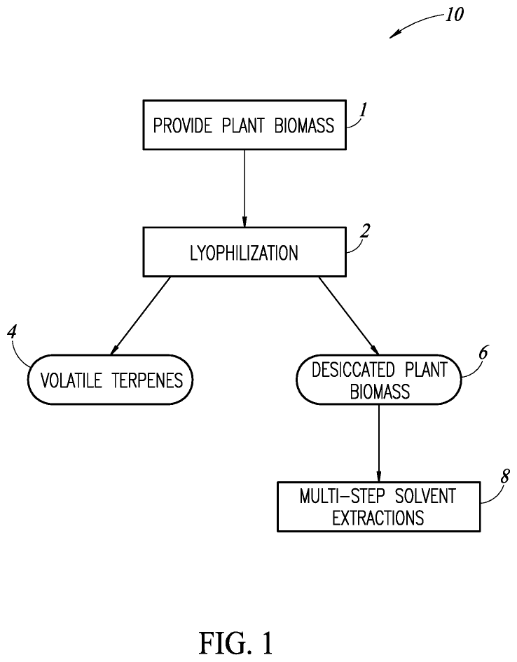

The process disclosed herein is capable of reproducibly producing complex chemovars from cultivars (e.g., cannabis) with minimal chemovar drift. Advantageously, the process effectively isolates the volatile and unstable/reactive chemical substances (e.g., terpenes) at high yields and produces multiple fractions of APIs that can be used alone or formulated into various end products. FIG. 1 shows a general flow chart of the process (10) comprising providing plant biomass (1), lyophilizing the plant biomass (2), which provides volatile terpenes (4) and desiccated plant biomass (6). The desiccated plant biomass (6) undergoes further multi-step solvent-based extractions (8).

1. Extracting Volatile Substances

In various embodiments, the process disclosed herein provides for extracting volatile substances as such terpenes from plant biomass. The plant biomass may be in any form, including fresh, dried, cured (dried over an extended period of time), frozen, or fresh-frozen forms. As discussed in more detail herein, frozen, especially fresh-frozen plant biomass is more conducive to preserving the live resins in the plant biomass.

One specific embodiment provides a process comprising providing fresh-frozen plant biomass; and lyophilizing the fresh-frozen plant biomass to provide desiccated plant biomass and a condensate including water and one or more volatile substances having a boiling point of no more than 250° C. at atmospheric pressure. Preferably, the fresh-frozen plant biomass may be reduced to a particulate form prior to lyophilizing. In various embodiments, the fresh-frozen plant biomass may be milled, grinded or otherwise mechanically separated into fine particles.

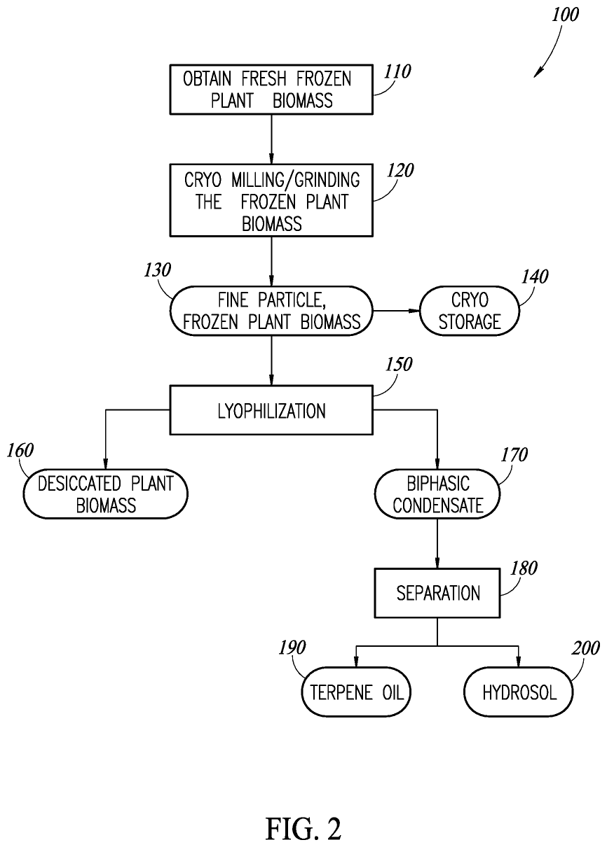

FIG. 2 shows the process according to a more specific embodiment. The process 100 includes obtaining fresh frozen plant biomass (110), cryo-milling or grinding the frozen biomass (120) to produce fine particles (130). The fine particles of frozen plant biomass can be packaged and stored in nitrogen purged bags (140); or be subject to lyophilization (150).

The lyophilization step is performed by controlled heat-vacuum-freeze cycles that sublimate solid water from the frozen biomass to produce desiccated plant biomass (160). Volatile substances such as terpenes are extracted along with water to form a condensate (170). The condensate is generally biphasic mixture including non-polar terpene oils (190) and an aqueous mixture of water and polar terpenoids and other hydrophilic substances such as plant saccharides, esters, phenols, etc. Advantageously, the volatile components of the plant biomass (i.e., those having boiling point of no more than 250° C. at atmospheric pressure) can be extracted and isolated with minimal loss or degradation. Less volatile chemical substances remain in the desiccated plant biomass (160) and can be subjected to further fractional extractions.

For cannabis, this first phase of extraction produces volatile terpene-enriched oils without extracting any cannabinoids. While the process, as described herein, is directed toward the extraction of plant biomass from the genus Cannabis, it should be noted that the methods provided can be readily adapted by those of ordinary skill in the art to extract active substances from other plant species, and this adaptability represents a further embodiment of the present disclosure.

The processes are described in more detail below.

a. Fresh-Frozen Plant Biomass

The sequential, multi-step process to selectively extract terpenes, along with other plant constituents, begins with fresh frozen plant biomass that is cryogenically processed to preserve the chemical constituents of the native plant.

The method disclosed herein is particularly suitable for processing plant matters such as cannabis, hemp and hop, in which the chemical substances to be extracted are generally concentrated in the delicate resinous flowers of the plants. For instance, although APIs such as cannabinoids are found in many parts of cannabis plants (including flowers, leaves and stalks), it is the resinous glands (i.e., trichomes) of the female flowers that produce the most amounts of cannabinoids, in addition to terpenes.

To prepare the plant biomass for extraction, whole flower cannabis is first harvested fresh from live plants. In doing so, the live plants are first striped of large water/fan leaves in the field to increase the concentration of APIs by mass in the harvested flower lots. Water leaves make up 15-20% of the plant and do not contain a viable concentration of API's. The fresh flowers are then separated or “bucked” by machine or by hand from the stems to increase the concentration of APIs by mass in the harvested flower lots. Stems make up approximately 10-12% of plant and do not contain a viable concentration of API's.

The fresh flowers are then immediately packed into nitrogen purged mylar bags, sealed and flash frozen. As used herein, flash freezing refers to a process whereby the plant biomass is rapidly frozen in a short period of time (within hours of harvest) and under cryogenic temperatures (e.g., lower than −18° C., or preferably lower than −30° C.). More specifically, the nitrogen-packed lot of fresh flower is flash frozen in freezers (e.g., at −34° C. or lower) or packed in dry ice at −75° C. Optimally the flowers are flash frozen within an hour of being harvested, which maximizes retention of terpenes and terpenoids.

Unlike the traditional cannabis drying and curing process, which invariably subjects the plants to conditions that induce loss or degradation of the volatile components such as terpenes, the freshly frozen plant biomass is prepared by avoiding heat, light, oxygen, or physical agitation. Because the plant is freshly frozen immediately following harvest and kept at freezing temperatures throughout the extraction process, the resulting extracts, also referred to as “live resin,” maintain its valuable terpene profile, thus retaining the plant's original flavor and aroma that can then be carried over into the end product formulated with terpenes.

The fresh frozen plant biomass (e.g., cannabis flowers rich in trichome glands) can remain at −24° C. to −34° C. during storage or transportation. It is important to maintain the “cold chain” to ensure that once the biomass is frozen it remains frozen until the lyophilization process.

b. Fresh-Frozen Plant Biomass as Fine Particles

In some embodiments, the fresh-frozen plant biomass may be reduced to fine particles under cryogenic conditions, a process also referred to as “cryo-milling,” prior to lyophilization. Advantageously, the cryogenic condition lowers the vapor pressure and keeping the volatile substances (such as terpenes) in a solid phase and entrained in the plant matrix. Additionally, the condition increases the brittleness of the plant biomass, making the milling process more efficient. Moreover, brittle materials absorb relatively little energy prior to fracture, which also minimize kinetic, thermal and chemical reactions. Cryo-milling thus continues to preserves the “live resin” of the plants.

To ensure the frozen plant biomass never defrosts, the fresh frozen plant biomass may optionally be first “crashed’ at an ultra-low temperature (e.g., in a −88° C. freezer).

The “crashed” plant biomass is subsequently introduced into a cutting mill along with dry ice or liquid nitrogen to maintain below-freezing temperatures (−60° C. to −210° C.) throughout the milling process.

To obtain uniform fine particles of the frozen plant biomass, the mill is fitted with a sieve cassette having appropriate perforation sizes that would determine the fine particle sizes. When the particles are at the desired sizes (e.g., no more than 10 mm, no more than 5 mm or no more than 1 mm), they are removed from the milling chamber by vacuum pressure, ensuring that residence time in the milling chamber is minimized. The cryo-milling process reduces and makes uniform the particle size of the plant material, and concomitantly increases the surface area. As a result of the reduction in particle size, the material's mass per volume ratio (density) is increased. In an extraction vessel of a fixed volume, a greater mass of plant material can be introduced, thereby increasing extraction throughput. Reduction of particle size to 1 mm or less is shown to increase mass transfer (e.g., extraction of API into a solvent system of subcritical or supercritical CO₂ or ethanol).

Alternative to milling, fresh frozen biomass may be mechanically separated to fine particles by agitating the fresh frozen plant biomass in a cold medium (e.g., nitrogen, dry ice, ice water, or a combination thereof) to create a trichome gland rich slurry. The slurry is then sieved through various nylon mesh filters to separate the trichome glands into micron-sizes (e.g., 30-120 microns).

The slurry may be further homogenized on a high shear homogenizer and or high-pressure homogenizer, resulting in cell lysis of trichome glands and expression of terpenes into the aqueous stationary phase.

The fine particles of the frozen plant biomass or the homogenized slurry may be directly subject to lyophilization; or may be packed in nitrogen purged containers (e.g., mylar bags) and stored in below-freezing temperature.

The sizes of the fine particles refer to the lengths of the longest dimension of given particle. Typically, the fine particles are larger than 1 micron and smaller than 30 mm in sizes. More specifically, at least 70%, or more typically, at least 85% of the frozen plant biomass by weight are particles within the above size range.

c. Lyophilization

“Lyophilization” or “freeze drying,” as used herein, refers to a process by which water is removed from the frozen plant biomass by sublimation, i.e., water is transformed from a solid form directly to a vapor form. Because below-freezing temperatures are employed in this process, degradation of the plant biomass and chemical substances contained therein is minimized. In particular, volatile substances, those that have boiling points of no more than 250° C. at atmospheric pressure, are also extracted from the plant biomass along with water.

To facilitate lyophilization, the plant biomass are evenly heated in a controlled manner. Although the plant biomass can be directly lyophilized in any form, frozen, or fresh-frozen plant biomass, especially when in the form of fine particles can facilitate with more efficient and even heating.

In a typical embodiment, the fresh, frozen or fresh-frozen plant biomass (e.g., as fine particles) are loaded in a heat-conductive container. Metal trays of aluminum or stainless steel tend to evenly and efficiently distributes thermal energy. Optionally and depending on the size of the container, additional metal ribs or dividers may be installed in the container to increase the surface area through the depth of the biomass.

The lyophilization may be carried out in freeze-dryers equipped with controllable heating and freezing elements, in addition to a vacuum pump. In a typical embodiment, one freeze-dry cycle may include 24 hours of sublimation or dry time at a temperature of no more than 65° C. and about 500 millitorr pressure, followed by about 4-9 hours of freeze time at about −40° C. More than one freeze-dry cycle may be needed to fully desiccate the plant biomass. More detailed description of controlling or optimizing the freeze-dry cycles may be found in U.S. Pat. No. 9,459,044, which is incorporated herein by reference in its entirety.

Once the desired moisture level is reached, preferably having no more than 2% water content, and more preferably no more than 1% water content, the desiccated plant biomass is removed and ready to be subject to further extractions (e.g., subcritical or supercritical CO₂ or ethanol extractions), or stored in a dry condition at about 4° C.

During freeze-drying, a condensate of water and volatile substances is withdrawn from the freeze dryer via vacuum pressure through an inline 25 μm hydrophobic filter and collected. Following the removal of the desiccated plant material, a thaw cycle is run for 1-2 hours; and the residual condensate in the interior of the freeze dryer is also collected. The condensate is a mixture of water, non-polar terpenes, polar terpenes, plant saccharides, esters and phenols and more. It can typically be seen as two distinct fractions (biphasic), with the non-polar terpene layer enriched with terpene oils float on top of an aqueous mixture of polar terpenoids, esters, phenols, and water. The aqueous mixture is also referred to as “hydrosol,” which contains water and polar volatile substances. Some water soluble materials dissolved in the water and other non-polar essential oil components may remain in the water phase as a colloidal suspension.

The condensate is stored under vacuum at below 5° C. before further refinement to isolate the terpenes. It is important to keep the condensate cold at all times to keep the volatile mono-terpenes in a liquid phase.

d. Fractional Freezing

The two liquid fractions of the condensate can be separated before further refinement to isolate the terpenes. The separation may be carried out by any convention means for liquid-liquid separation, including for example, by a separatory funnel.

The hydrosol (which is the lower phase) is first released by gravitation, if using a separatory funnel. The hydrosol is stored for further use as a liquid ingredient or as a key component in hydrosol ice polishing step, as described herein.

The non-polar volatile terpene fraction, which is cannabinoid-free and enriched with terpene oils, is crashed to −70° C. to freeze out any remaining/unseparated hydrosol. The terpene fraction may optionally be filtered under vacuum pressure through a polyethersulfone (PES) membrane (pore size 0.22 μm) at −34° C. to separate the liquid phase (terpene oils) from the solid phase (frozen hydrosol) and to ensure filtration removal of microbial contaminants. Following membrane filtration, the refined terpene fraction should be stored at low temperature (e.g., no more than −24° C.).

2. Subcritical or Supercritical Carbon Dioxide (CO₂) Multi-Phase Extraction (MPE)

In a further embodiment, the process further comprises contacting the desiccated plant biomass with subcritical or supercritical CO₂ under conditions sufficient to sequentially extract a first fraction of terpenes; a second fraction of neutral cannabinoids; and a third faction of acidic cannabinoids.

Subcritical or supercritical CO₂ is a non-toxic solvent, the solvency of which can be adjusted by increasing/decreasing temperature and/or pressure. Typically, CO₂ is considered supercritical at temperature about 88° F. and pressure of 1083 psi. At below 88° F., CO₂ is subcritical.

The extraction is thus carried out sequentially by adjusting the temperatures and pressure of CO₂, contacting time, and run time. Different cultivars may require different settings to maximize the efficiency of separation between different fractions.

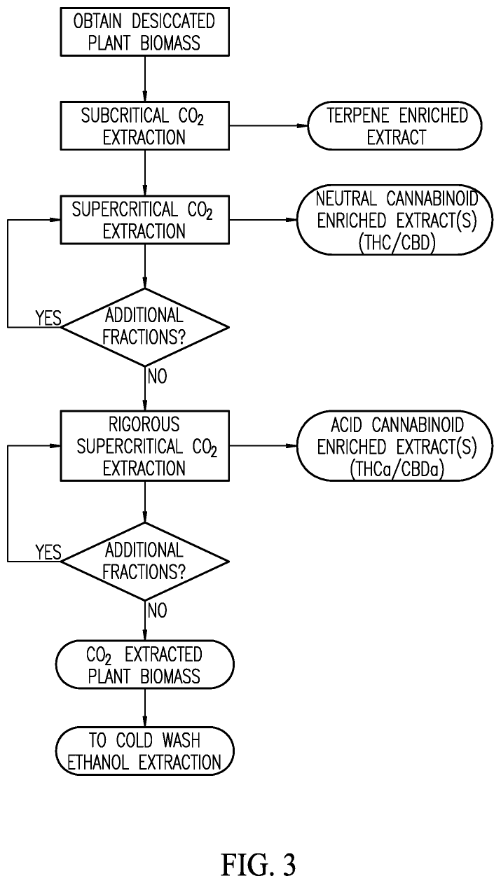

Depicted in FIG. 3 is a flow chart according to a more specific embodiment. As shown, the process comprises obtaining and loading desiccated plant biomass into extraction column, contacting the same with subcritical CO₂ to obtain a terpene-enriched extract; followed by extracting neutral cannabinoids-enriched extracts (THC and CBD) under supercritical condition; followed by extracting the acidic cannabinoids under more rigorous supercritical condition. After the completion of the CO₂ extraction, the remaining biomass can be subjected to cold wash ethanol extraction.

These steps are described in further detail below.

a. Terpene Fraction

The desiccated plant biomass is packed tightly into extraction column. An impact hammer may be used to increase the compactness and density of the fine particles within a finite volume. Extracting more mass per batch enables more utility out of the primary cannabinoid extraction system.

Terpenes, including residual volatile terpenes and heavier, less volatile terpenes can be extracted by CO₂ at a subcritical phase. The first fraction targeted is mono and sesquiterpene rich with minimal cannabinoids (no more than 40% THC and CBD). The fraction may be collected after 1 hour of run time and stored at −24° C. for formulation later.

b. Neutral Cannabinoid Fraction

The neutral cannabinoid fractions contain THC and/or CBD. The CO₂ extractor is set to operate in the supercritical range. Fractions are collected based on the quality of the plant biomass and parameters of the run. The neutral fraction is typically collected after 1 hour of run time, then stored at −24° C. for further refinement.

c. Acidic Cannabinoids Fractions

The CO₂ extractor is set to operate in the more energy intense ranges (higher pressure and temperature) of the supercritical phase and run times extended. By increasing the total energy in the solvent system the total solubility index increases, which enables efficient extraction of the acidic cannabinoids such as THCA and CBDA.

Acid rich cannabinoid fractions are kept separately from the terpene and neutral fractions to minimize super saturation points of acid cannabinoids in the neutral fractions. If the super saturation point of acid cannabinoids is reached in the “oil fractions” THCA will precipitate out of the solution and crystalize which increases viscosity and causes dysfunction in the vapor cartridge hardware.

The acid rich fractions are well suited for tactile, crystalline, “dab” products that are of higher potency. By fractionating the terpenes (by both lyophilization and CO₂ MPE) and neutral cannabinoid by CO₂ MPE, the super saturation points of THCA are increased and subsequent crystallization of THCA makes for a 90% plus pure THCA product.

3. Ethanol (ETOH) Extraction

The desiccated plant biomass obtained after volatile terpene removal may also be subjected to ETOH extraction, as an alternative to supercritical or subcritical CO₂ extraction. The extracts may be used as Edible Cannabis Oil (ECO), “Dabs,” vape oil, topical oil, distillate and isolates, all of which are cannabis concentrate inhalation extracts.

The particle sizes of the desiccated plant biomass should be controlled during cyro-milling to be uniform and fine yet above micron sizes. The goal is avoid having particles pass through the micron-sized pores in the mesh of the extraction bag. This will ensure minimal vegetative solids contaminate the crude extract. If vegetal material makes it past the mesh extraction bag, it can carry color bodies such as chlorophyll, anthocyanins, caternoids etc. in its cellular structure. Many of the color bodies in the plant biomass are bitter and impart undesirable flavors if the extracts are inhaled and/or ingested.

Thus, particle size control during cryo-milling can improve the clarity, color and potency of the ethanolic tincture. If the milling is poorly done then the vegetal mass can pass the filtration of the extraction bag. Overtime, especially if temperatures of the ethanolic tincture rise above freezing the color bodies will elute into the ETOH crude and contaminate the extract. This will cause a need for further refinement to remove the contaminants.

The color body constituents, on the other hand, can remain in the plant matrix if the solvent is kept below −34° C. and the residence times are controlled. The primary ETOH extraction is thus performed under −34° C. to −88° C. to minimize extracting polar substances that can result in color body contamination.

4. Post-Processing

A number of post-processing steps may be carried out to further refine the crude extracts obtained according to the processes disclosed herein. Conventional means such as filtration, chromatography, distillation, activated carbon and other media can be used to remove contaminants or further separate the chemical substances in the crude extracts.

Crude CO₂ fractions may be separated by qualitative analysis. The viscosity of the crude extract at ambient temperatures will indicate the neutral and acid rich fractions. Terpenes and neutral cannabinoids (THC and CBD) fractions are of relatively low viscosity and can be set aside for vapor oil. Acid rich fractions are not targeted and left in the plant matrix for secondary extraction via ETOH.

a. Hydrosol Polishing

Hydrosol may be further processed by ice polishing to reconstitute the polar terpenoids and plant saccharines in the hydrosol fraction back into the crude CO₂ extract. The ice-polishing process can also extract polar compounds co-extracted in primary CO₂ extraction into the polar phase to sublimate out.

More specifically, the hydrosol fraction obtained from the lyophilization is frozen, e.g., into ice cubes. The crude CO₂ extract is crashed to ultra-low temps of less than −70° C. The crude extract is then homogenized w/ dry ice and hydrosol ice and milled into a fine frozen powder. The powder is lyophilized at 43° C. to cause the sublimation of the polar components.

b. Winterization

Winterization is another post-processing step that precipitates waxes, lipids and other polar constituents from an ethanolic solution where ETOH is the reagent used to dilute crude cannabis extract. More specifically, terpene rich CO₂ fractions are blended with neutral fractions and diluted at 10:1 ratio with ETOH. The solution is homogenized via high shear mixing and ultrasonic energy in an ultrasonic bath at 50° C. for 1 hour. The ETOH crude is then transferred to a glass container (e.g., a carboy) and crashed to −88° C. overnight to allow for precipitation of non-APIs such as wax and lipids.

The chilled mixture undergoes vacuum assisted 25 um filtration at a temperature of −34° C. or lower to produce a primary filtrate. The primary filtrate is again cooled to nominally −88° C. overnight to precipitate additional undesirable coextractants. The chilled mixture undergoes vacuum assisted 10 um filtration at a temperature of −34° C. or lower to produce a secondary filtrate. This secondary filtrate is treated with a mixture of activated carbons, mixed and then heated in an ultrasonic bath at 50° C. for 1 hour. The carbon treatment captures color bodies and contaminants that have remain in solution following the previous winterization steps.

The carbon treated secondary filtrate is then passed through a column of silica gel. Said column is prepared as a filter aide by creating a slurry of ethanol and silica gel and using standard chromatographic column packing techniques, taking care not to dry out the silica gel. The carbon treated ethanol is poured over the silica gel column with vacuum pressure applied to approximately 5,000-10,000 micron. The first elution is collected after 10 mins and recycled into the bulk, treated mixture. The tertiary filtrate once collected, is cooled to nominally −88° C. overnight to precipitate additional undesirable coextractants. The chilled mixture undergoes vacuum assisted 1 μm filtration at a temperature of −70° C. or lower to produce a quaternary filtrate. The quaternary filtrate is again cooled to nominally −88° C. overnight to precipitate additional undesirable coextractants. The chilled quaternary filtrate undergoes vacuum assisted 0.22 μm filtration at a temperature of −70° C. or lower to produce the final winterized extract that is then concentrated by removing ethanol by distillation using a rotary evaporator.

The ethanol is distilled away using a rolling film method to a final concentration of approximately 15-25% by mass. The ethanol that is collected is of sufficient quality to be reused for subsequent winterization procedures.

c. Concentration

In still another embodiment, winterized and volume reduced combined terpene enriched and neutral, THC and/or CBD containing extracts are further processed to reduce additional volume (e.g., solvents) and arrive at a product that has the viscosity and composition amenable to final product formulation. The solvent reduction or purge can be carried out by any means, including heating or rotary evaporation, optionally under vacuum. Preferably, the residual ethanol content after purge is less than 500 ppm, or more preferably, the residual ethanol content after purge is less than 1 ppm.

In one embodiment, the volume reduced combined extract is poured from the evaporating flask into trays made from silpat material covered with PTFE lined parchment paper. The product is spread across as many trays as possible maximizing surface area and minimizing the depth of the pool/film. The depth of the product pool is important for purge time optimization and purge efficiency. Generally the thinner the pool and the more surface area exposed the better. The material is placed into a vacuum oven and set to <500 microns at 30-40° C. for no less than 100 hours. During the drying process, the material is folded and thoroughly mixed every 24 hours into a homogenous mass and then spread thin. The product, a THC and/or CBD rich oil, is purged of ethanol down to less than 500 ppm. The refined material is stored protected from light under vaccum, or covered in an inert gas (e.g. nitrogen and/or argon), at temperatures between 4 and 8° C. until needed for formulation.

In yet another embodiment, winterized and volume reduced combined quick wash ethanol and vigorous supercritical CO₂ THCA and/or CBDA containing extracts are further processed to reduce additional volume and arrive at a product that has the viscosity and composition amenable to final product formulation. The volume reduced combined extract is poured from the evaporating flask into trays made from silpat material covered with PTFE lined parchment paper. The depth of the product pool is important for ideal crystal formation, generally the deeper the pool the better but no deeper than a few inches. The tray is covered with parchment paper and set on a thermal cycling heating pad. The pad is set to cycle between 30 and 40° C. until nucleation occurs (24-100 hrs). After nucleation is completed the product is placed into a vacuum oven and set to <500 microns at 30-40° C. for no less than 100 hours. The product, a THCA and/or CBDA rich semi-solid, is purged of ethanol down to less than 500 PPM. The refined material is stored protected from light under vacuum, or covered in an inert gas (e.g. nitrogen and/or argon), at temperatures between 4 and 8° C. until needed for formulation.

In certain embodiments the product semisolid is a THCA-rich material, containing 70-90% THCA.

### Formulating End Products

The isolated fractional extracts can be formulated into various end products (e.g., terpenes, vape oils, dabs, etc), which maintain or resemble the flavor, aroma and therapeutic profiles of the cultivar, from which the fractional extracts are obtained.

1. Inhalation Products

“Inhalation extracts” are Cannabis extracts produced for consumption through vaporizing the material and inhaling it. There are generally two forms of inhalation extracts (IE): “vape oil” and dabs.

Vape oil is a liquid oil formulation, frequently containing a higher proportion of neutral cannabinoids (e.g. THC and/or CBD), and often mixed with flavor and aroma components, such as terpenes. Vape oil is designed for use in hand held, vaporizing devices such as vape pens. Typically, the vape oil is provided in a specially designed cartridge.

Live resin vape oils are generally formulated by combining cannabis derived terpenes (CDT) with neutral cannabinoids (THC and/or CBD). CDT may be obtained from lyophilization and/or terpene fraction from CO₂ SPE, or ethanol extraction, as described herein. Depending on the viscosity requirements for vapor hardware, terpenes are mixed into the final mass and fully homogenized at about 5-30% by extract mass.

“Dabs” or “dabbables” are viscous, high concentration, acid-rich cannabis extracts (THCA) that are smoked by heating the product (e.g., on a hot surface), which rapidly vaporizes. During dabbing, THCA is thermally converted into THC. CDT can be combined with THCA-rich fraction.

Methods for creating the formulations in the preceding embodiments include warming the THC, THCA, CBD and/or CBDA rich oils on a hot pad to no more than 40° C. for oil formulations and 40-60° C. for Dab formulations. Once heated, terpenes are mixed into the final mass at 5-30% by extract mass for oil formulations and 5-15% by extract mass for Dab formulations. The oil/terpene mixtures are homogenized and then cooled to lower than 5° C. to prevent loss of volatile components.

2. Edibles

The ethanolic or CO₂ extracts and concentrations disclosed herein may be formulated into edibles for ingestion. The common forms of edibles include beverages, baked goods, cooling oil, tinctures, etc. The formulation and dosing are known to a skilled person in the art.

3. Topical Formulations

The ethanolic or CO₂ extracts and concentrations disclosed herein may be formulated for topical application. The topical formulation may be in the form of oil, lotion, ointment, spray, cream, etc., and may be applied directly to the skin (including the scalp). Because CB1 receptors are expressed in the epidermal layers, muscles and nerves, cannabinoids such as THC can bind directly to CB1 receptors and provide localized relief to inflammation or pain. The cannabinoids may also enter into the bloodstream through transdermal action and further interact with CB2 receptors.

### EXAMPLES

### Example 1

### Volatile Terpene Extraction by Lyophilization of Milled Plant Biomass

Into each of five customized aluminum trays was evenly distributed about 1000-1500 grams of cryo-milled flash frozen Juicy Fruit cannabis strain. The trays were loaded into a Harvest Right freeze dryer and subjected to freeze drying cycles of 24 hours of sublimation or dry time at a temperature of no more than 65° C. and about 500 millitorr pressure, followed by about 4-9 hours of freeze time at about −40° C.

When the end of the cycle had been indicated by the instrument, the desiccated material was removed from the oven and a thaw cycle initiated on the condenser for 2 hours at 65° C. to thaw the accumulated material that had collected. The resulting biphasic condensate is removed via vacuum pump through an inline 25 um hydrophobic filter and transferred to a separatory funnel. When clear separation of the layers was evident, the lower aqueous hydrosol layer (approximately 800 mL) containing polar terpenoids and other polar, volatile organic materials was removed and saved for later use. The non-polar, organic phase (approximately 150 mL) was removed from the separatory funnel and stored at approximately −70° C. for 4 hours to freeze out any remaining hydrosol. This mixture was vacuum filtered through a 0.22 um PES membrane at −34° C. to separate the refined terpenoid extract from frozen hydrosol. The terpene rich extract is stored under vacuum at temperatures of −24° C. or less.

The desiccated plant biomass can be stored at −4° C. to ensure material stability and to minimize rehydration, or can be taken directly to subsequent multiphase CO₂ extraction.

### Example 2

### Multiphase CO₂ Extraction of Desiccated Plant Biomass

Into the extraction chamber of an Eden Labs 20L Hi-flow CO₂ extraction instrument was placed 9000 g of desiccated Juicy Fruit material from the lyophilization procedure described above. The material was carefully packed tightly into the column using a modified impact hammer. The accumulator pressure was set for liquid subcritical operation at 1000 psi and the extraction was allowed to proceed for 30 mins at a temperature of 10 C. The terpene rich fraction was removed from the collection vessel and stored at −24° C. for later refinement and formulation.

Without making any changes to the material in the extraction chamber, the extraction instrumentation was set to operate in the supercritical range to target neutral cannabinoids such as THC and CBD. The extraction vessel pressure was set at 1800 psi and the extraction was allowed to proceed for 3600 mins at a temperature of <37° C. The extract was collected and stored at −24° C. until it was further refined.

Again, without making any changes to the material in the extraction chamber, the extraction instrumentation was set to operate in a more rigorous supercritical range to target acid cannabinoids, such as THCA and CBDA. The extraction vessel pressure was set at 2000 psi and the extraction was allowed to proceed for 6000 minutes at a temperature of <37° C. The extract was collected and stored at −24° C. until it was further refined.

### Example 3

### Quick-Wash Ethanol Extraction of Desiccated and Terpene Extracted Plant Biomass

The CO₂ extracted plant biomass material (4500 g) was placed into a 25 micron pore extraction bag contained within an extraction centrifuge. Cold ethanol (95%, 56 L, −40° C.) was added and the extraction was allowed to proceed for 20 min at −34 to −88° C. It is important to keep the extraction cold at all times to minimize the extraction of colored bodies (i.e. chlorophyll, anthocyanins, caternoids) that impart undesirable properties to the extracts and may necessitate additional refinement.

### Example 4

### Terpene Analysis— Comparing Extraction Methods

Samples of the Tesla Tower Cannabis cultivar were obtained and subjected to extraction and analysis for terpene composition. Complete extracts and raw materials were analyzed using gas chromatography/mass spectrometry (GC/MS) to provide detailed analysis of the terpene content including individual species and their relative ratios.

Three samples were analyzed. The first included analysis of the input flower material to determine the native terpene chemovar, whereby a baseline chromatographic fingerprint of terpene composition of the material was determined. The second sample used a hydrocarbon extraction method to create a terpene extract that suffers loss of terpene due to conditions of the extraction. The third sample was produced using the extraction methods of the present invention, as described herein.

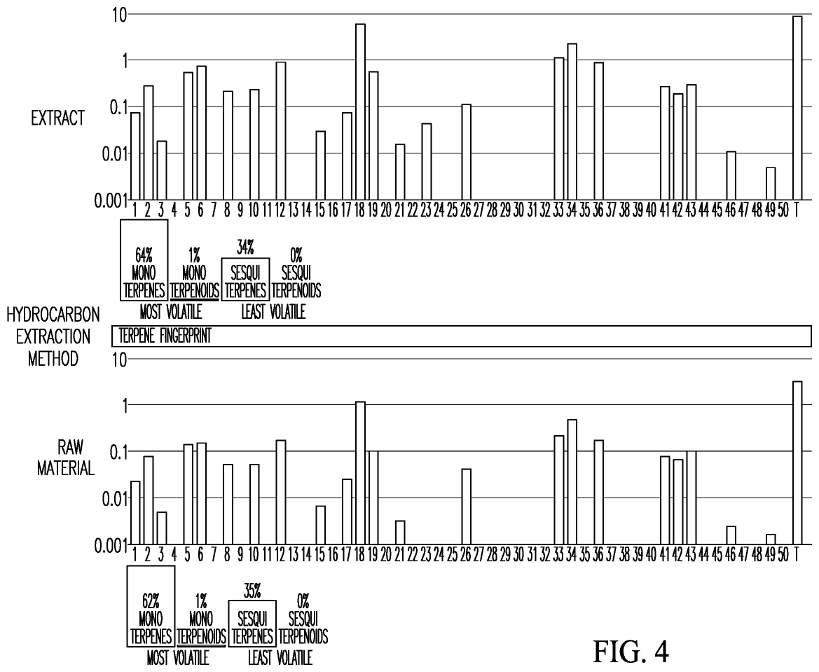

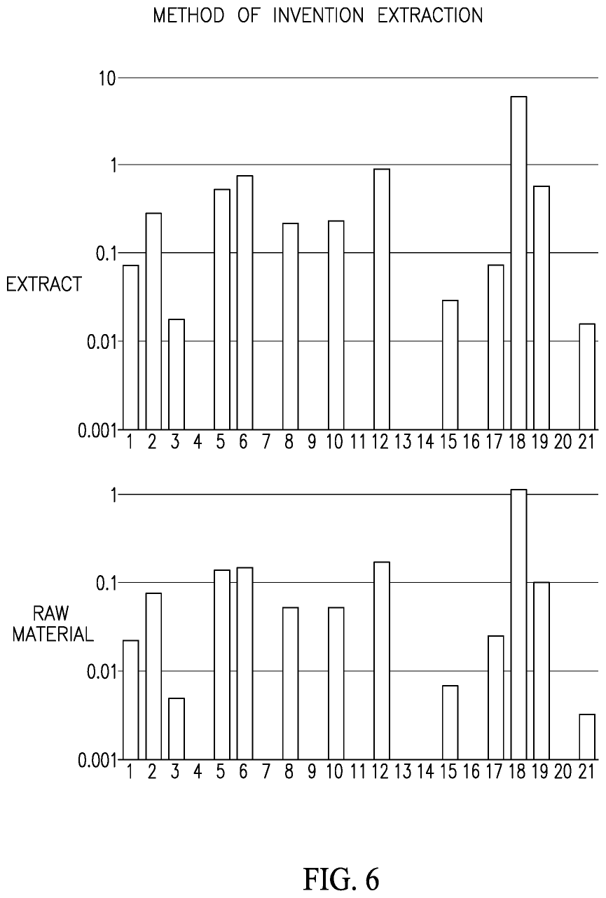

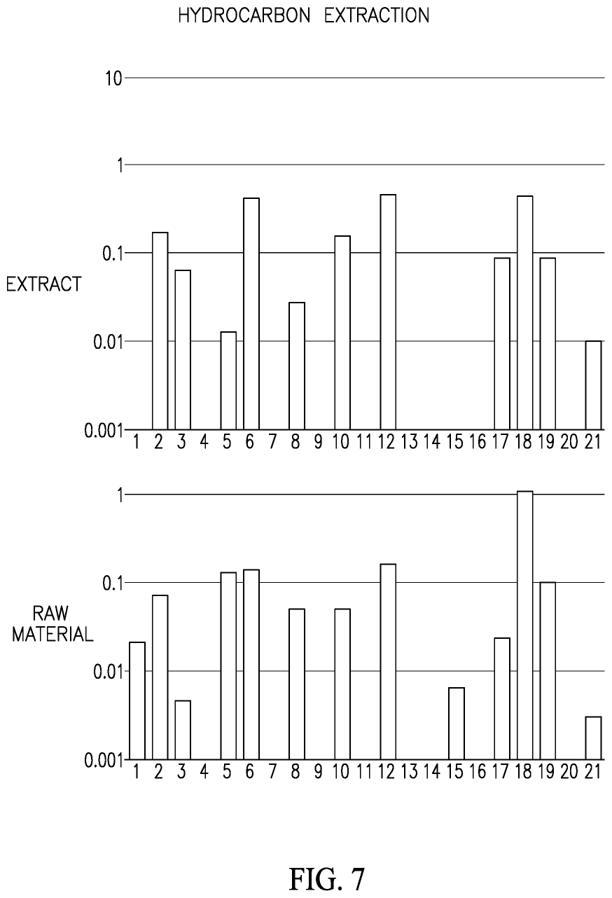

FIGS. 4-7 show bar-chart modified chromatographic representations for the GC/MS analyses of extracts described above. Notably, the sample derived from the extraction methods of the present invention maintains a very similar terpene fingerprint to that obtained from the raw plant material.

Table 1 shows the side-by-side comparison of the various concentrations of terpenes present in the extracts and the raw material, as depicted in FIG. 4.

TABLE 1

| | | | Concentration |
|---|---|---|---|
| | Chemical | Concentration | in Raw |
| Reference # | Name | in Extracts (%) | Material (%) |
| 18 | Terpinolene | 6.2 | 1.1 |
| 34 | Caryophyllene | 2.3 | 0.44 |
| 33* | Farnesene | 1.2 | 0.21 |
| 36 | Humulene | 0.94 | 0.16 |
| 12 | Limonene | 0.92 | 0.16 |
| 6 | Myrcene | 0.77 | 0.14 |
| 19 | trans-Ocimene | 0.58 | 0.099 |
| 5 | β-Pinene | 0.57 | 0.13 |
| 43* | Selinadiene | 0.3 | 0.092 |
| 2 | α-Pinene | 0.29 | 0.072 |
| 41* | β-Maaliene | 0.27 | 0.069 |
| 10* | α-Terpinene | 0.24 | 0.05 |
| 8* | Carene | 0.23 | 0.049 |
| 42* | Pseudo-Valencene | 0.19 | 0.061 |
| 26 | Terpineol | 0.12 | 0.039 |
| 1* | Thujene | 0.079 | 0.021 |
| 17 | g-Terpinene | 0.077 | 0.024 |
| 23 | Fenchol | 0.044 | — |
| 15* | Eucalyptol | 0.031 | 0.0064 |
| 3* | Camphene | 0.018 | 0.0046 |
| 21 | Linalool | 0.016 | 0.0031 |
| 46 | Caryophyllene Oxide | 0.011 | 0.0023 |
| 49 | Bisabolol | 0.0051 | 0.0015 |
| T | Total | 15 | 2.9 |

*Compounds identified using a spectral library

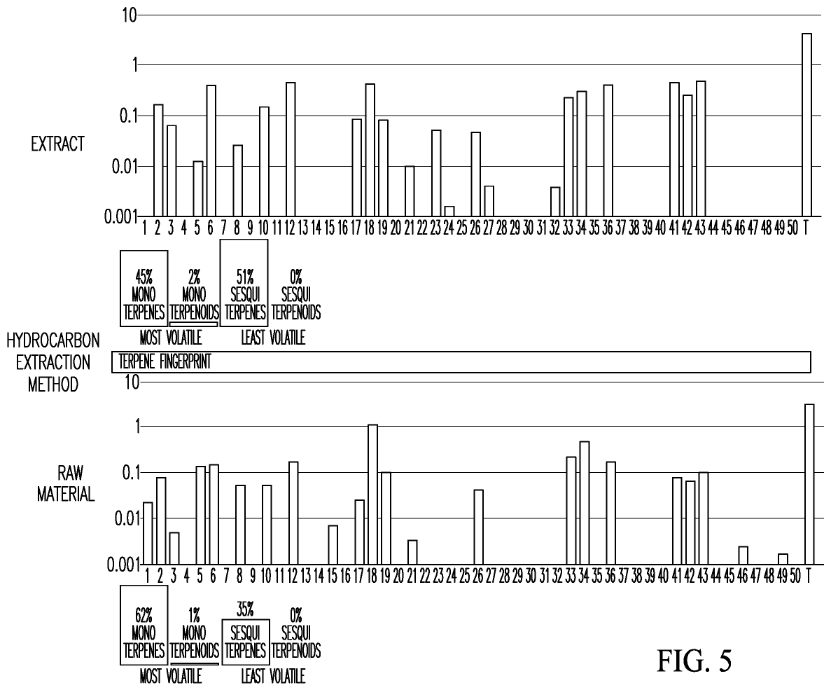

Table 2 shows the side-by-side comparison of the various concentrations of terpenes present in the extracts and the raw material, as depicted in FIG. 5.

TABLE 2

| | | | Concentration |
|---|---|---|---|
| | Chemical | Concentration | in Raw |
| Reference # | Name | in Extracts (%) | Material (%) |
| 43* | Selinadiene | 0.47 | 0.092 |
| 12 | Limonene | 0.43 | 0.16 |
| 41* | β-Maaliene | 0.42 | 0.069 |
| 18 | Terpinolene | 0.4 | 1.1 |
| 6 | Myrcene | 0.39 | 0.14 |
| 36 | Humulene | 0.38 | 0.16 |
| 34 | Caryophyllene | 0.29 | 0.44 |
| 42* | Pseudo-Valencene | 0.24 | 0.061 |
| 33* | Farnesene | 0.22 | 0.21 |
| 2 | α-Pinene | 0.15 | 0.072 |
| 10* | α-Terpinene | 0.14 | 0.05 |
| 17 | g-Terpinene | 0.08 | 0.024 |
| 19 | trans-Ocimene | 0.078 | 0.099 |
| 3* | Camphene | 0.058 | 0.0046 |
| 23 | Fenchol | 0.05 | |
| 26 | Terpineol | 0.045 | 0.039 |
| 8* | Carene | 0.026 | 0.049 |
| 5 | β-Pinene | 0.012 | 0.13 |
| 21 | Linalool | 0.0096 | 0.0031 |
| 27 | Geraniol | 0.0038 | |
| 32* | Thymol | 0.0037 | |
| 24 | Isopulegol | 0.0015 | |
| 1* | Thujene | | 0.021 |
| 15 | Eucalyptol | | 0.0064 |
| 46 | Caryophyllene Oxide | | 0.0023 |
| 49 | Bisabolol | | 0.0015 |
| T | Total | 3.9 | 2.9 |

*Compounds identified using a spectral library

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet including U.S. Provisional Patent Application No. 62/925,137, filed on Oct. 23, 2019, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A process comprising:
providing plant biomass; and
lyophilizing the plant biomass to provide desiccated plant biomass and a condensate including water and one or more volatile substances having a boiling point of no more than 250° C. at atmospheric pressure.

2. The process of claim 1 wherein the plant biomass is cannabis, hemp or hop.

3. The process of claim 1, wherein the plant biomass comprises cannabis flowers.

4. The process of claim 1, wherein the plant biomass is fresh, cured, or frozen plant biomass.

5. The process of claim 4, wherein the frozen plant biomass is fresh-frozen plant biomass by flash freezing fresh plant biomass.

6. The process of claim 5 wherein the fresh-frozen plant biomass is in the form of fine particles.

7. The process of claim 6 wherein forming the fine particles of the frozen plant biomass comprises milling or grinding at below-freezing temperature.

8. The process of claim 1 wherein lyophilizing the plant biomass comprises running one or more cycles of sublimation and freezing, each cycle including:
sublimating solid water in the fine particles of the frozen plant biomass under vacuum and heating to a temperature of no more than 65° C. to provide sublimated plant biomass; and
freezing the sublimated plant biomass,
wherein the cycle is repeated till the sublimated plant biomass has a moisture level of no more than 2% w/w, thereby providing the desiccated plant biomass.

9. The process of claim 1, wherein the condensate includes non-polar terpenes, and an aqueous mixture having polar terpenes, plant saccharides, esters, phenols or combination thereof.

10. The process of claim 9 further comprising isolating the non-polar terpenes from the aqueous mixture.

11. The process of claim 1, further comprising contacting the desiccated plant biomass with supercritical or subcritical CO₂ under conditions sufficient to sequentially extract a first fraction of one or more terpenes; a second fraction of neutral cannabinoids; and a third faction of acidic cannabinoids.

12. The process of claim 11 wherein the neutral cannabinoids are THC, CBD or a combination thereof.

13. The process of claim 11 wherein the acidic cannabinoids are THCA, CBDA or a combination thereof.

14. The process of claim 1, further comprising contacting the desiccated plant biomass with ethanol under conditions sufficient to extract neutral or acidic cannabinoids from the desiccated plant biomass.

15. The process of claim 14 further comprising removing a non-active ingredient selected from the group consisting of wax and lipid.

16. The process of claim 1, further comprising combining at least two of the following fractions:
non-polar terpenes produced by lyophilization,
polar terpenes produced by lyophilization;
plant saccharides produced by lyophilization,
one or more terpenes produced by extraction;
neutral cannabinoids produced by extraction; and
acidic cannabinoids produced by extraction.

17. The process of claim 16 further comprising removing residual water or ethanol.

18. A plant-extract composition produced by the process of claim 16.

19. The plant-extract composition of claim 18 in an inhalable, edible or topical formulation.

20. The process of claim 11 further comprising removing a non-active ingredient selected from the group consisting of wax and lipid.
